(19) United States
(12) Patent Application Publication
(10) Pub. No.: US 2019/0108915 A1
(43) Pub. Date: Apr. 11, 2019

(54) DISEASE MONITORING FROM INSURANCE CLAIMS DATA

(71) Applicant: IQUITY, INC., Nashville, TN (US)

(72) Inventor: Charles Floyd Spurlock, III, Nashville, TN (US)

(21) Appl. No.: 16/152,861

(22) Filed: Oct. 5, 2018

Related U.S. Application Data
(60) Provisional application No. 62/568,739, filed on Oct. 5, 2017.

(51) Int. Cl. G16H 50/30; G06Q 40/08; G16H 50/20; G06Q 10/10; G06N 20/10; A61K 31/4704; A61K 31/277; C07K 16/28; A61K 31/137; A61P 25/28

(52) U.S. Cl. CPC G16H 50/30; G06Q 40/08; G16H 50/20; G06Q 10/10; G06N 20/10; A61P 25/28; A61K 31/277; C07K 16/2866; A61K 31/137; C07K 16/2887; A61K 31/4704

(57) ABSTRACT

The invention provides methods for identifying a disease status in a patient from claims data. A machine learning algorithm may be trained to report that a given patient is possibly affected by a disease, and the machine learning algorithm may be able to do so long before disease symptoms manifest to a problematic degree. The machine learning algorithm may be able to give an early warning that a patient is at a high risk of disease based principally on inputs provided in the form of insurance claims data.

### RELATED APPLICATION

The present application claims the benefit of and priority to U.S. provisional patent application Ser. No. 62/568,739, filed Oct. 5, 2017, the contents of which are incorporated by reference.

### TECHNICAL FIELD

The disclosure relates to identifying and treating diseases in patients.

### BACKGROUND

While the understanding of disease has expanded greatly in recent decades, there are still many serious diseases that the medical community is ill-equipped to diagnose and treat. Many of those diseases would exhibit improved outcomes if detected and treated early. Unfortunately, detecting a disease has historically followed a paradigm in which a patient seeks help from a medical provider when the patient experiences problems or symptoms that trouble the patient. For example, a patient may notice some dizziness or shortness of breath, and then observe over time that those symptoms appear to be aggravated. At some point, that patient may go see a doctor to see if there is a disease. However, in many cases, when the symptoms have advanced to such a degree, so too has the disease, and treatment options are limited.

### SUMMARY

The invention provides methods for identifying a disease status in a patient from claims data. A machine learning algorithm may be trained to report that a given patient is possibly affected by a disease, and the machine learning algorithm may be able to do so long before disease symptoms manifest to a problematic degree. The machine learning algorithm may be able to give an early warning that a patient is at a high risk of disease based principally on inputs provided in the form of insurance claims data. The insurance claims data may include patterns of diagnoses, treatments, hospital and doctor visits, as well as demographic and geographic data in which latent patterns are predictive of disease risk. The machine learning algorithm discovers patterns within training data sets in which the training data includes historical claims data as well as known disease outcomes. The machine learning algorithm may potentially identify a patient at a high risk of disease long before the risk would be discovered by a patient him- or herself, or in the course of routine doctor visits.

Not only may the machine learning algorithm identify a disease status (e.g., “high risk”) from claims data, the machine learning algorithm may characterize a level of activity of the disease in the patient, stratify the patient by severity, and correlate the disease status to efficacious treatment regimes. The machine learning algorithm may play important roles in monitoring, for recurrence or compliance, by correlating patterns in the claims data to patterns of treatment compliance or disease recurrence/remission.

Additional factors may be included in disease analysis including medical history and social factors such as demographic information, environmental considerations, patient or family history of disease, smoking, drug use, exercise, socio-economic information, and patient height, weight, or body mass index. Any of the above additional factors may be combined with insurance claim data to diagnose or monitor disease states. Many of the above additional factors may be determined from insurance claims data. By combining data related to the above additional factors with known outcomes for patients, patterns may be identified through, for example, machine learning analysis, to link combinations various data points to various outcomes such that subsequent identification of those patterns in new patients may be indicative of the linked outcome for the new patient.

Other factors that may be included in training sets and subsequent diagnostic and prognostic models may include imaging analysis such as histological analysis of patient body fluid or tissue samples and other more standard diagnostic techniques. Any patient-specific information may be provided for analysis, including genetic analyses, body fluid analyses, tissue biopsies, and other medical information. The more data that is provided to machine learning algorithms of the invention, the more possible patterns can be identified and, accordingly, diagnostic and prognostic analyses using said algorithms are more accurate and sensitive.

Because systems and methods of the invention can give an early warning that certain patients are at a high risk of a disease, physicians have the opportunity to intervene very early and treat a disease early or even prophylactically. Because systems and methods may be used to stratify patients based on disease activity or severity, treatment may be selected that will be effective, and poor treatment choices are avoided. Because systems and methods are useful for monitoring treatment and compliance, long term outcomes will be consistently improved.

Analytical devices, such as biosensors may be used to collect, monitor and convey physiological data using the systems and methods described herein. In some embodiments of the invention, analytical devices may be used for conveying diagnostic or prognostic information determined using the systems and methods described herein. In certain embodiments, methods such as color coded reporting may be used for conveying diagnostic or prognostic information determined using the analytical systems and methods described herein. In order to simplify diagnostic information, specific codes that are indicative of suggested action may be used. Physiological, diagnostic and prognostic information collected by the analytical device may be analyzed with, for example, claim data, to monitor or track identified patterns or signals over time and provide alerts when various thresholds are passed.

In certain aspects, the invention provides a treatment support method. The method includes training a machine learning algorithm on a training data set that includes historical claims data and known outcomes, providing claims data for a patient, and identifying—by the machine learning algorithm—a disease status for the patient. Identifying the disease status may include identifying the patient as being at a high risk for a disease. Preferably, the machine learning algorithm is implemented in a computing system comprising at least one processor coupled to a tangible, non-transitory memory subsystem. Optionally, identifying the disease status includes classifying an activity level of a disease in the patient.

The method may include recommending a treatment for the patient. Moreover, the method may include administering the treatment to the patient.

In an exemplary embodiment, the disease is multiple sclerosis (MS), and the activity level is selected from the group consisting of low, middle, and high, and when the activity level is low, the treatment includes the administration of laquinimod or terifunomide; when the activity level is middle, the treatment includes the administration of daclizumab, fingolimod, DMF, or ocrelizumab; and when the activity level is high, the treatment includes the administration of ocrelizumab, natalizumab, mitoxantrone, or alemtuzumab.

In some embodiments, identifying the disease status includes determining a therapeutic efficacy of a treatment. Identifying the disease status may include determining a disease progression. The disease may be a neurological disease, an inflammatory disease, a rheumatic disease, or an autoimmune disease.

Training the machine learning algorithm may include providing the training data set to the machine learning algorithm and optimizing parameters of the machine learning algorithm until the machine learning algorithm produces output describing the known outcomes.

The machine learning algorithm may include a neural network, a random forest, Bayesian classifier, logistic regression, decision tree, gradient-boosted tree, multilayer perceptron, one-vs-rest, and Naive Bayes, a support vector machine (SVM), or a boosting algorithm. In some embodiments, the machine learning algorithm includes a random forest comprising a plurality of decision trees. The decision trees receive parameters such as: icd codes; cpt codes; HCPCS codes; patient demographic data; and patient geographic data. In certain embodiments, the machine learning algorithm includes a neural network.

The disease may be Parkinson's disease, Alzheimer's disease, and epilepsy, Crohn's disease, ulcerative colitis, and IBD (inflammatory bowel disease), systemic lupus erythmatosus, rheumatoid arthritis, or fibromyalgia.

### BRIEF DESCRIPTION OF THE DRAWINGS

FIG. 1 diagrams a method.

FIG. 2 a system of the invention

FIG. 3 shows a machine learning system discovering associations in the data.

FIG. 4 shows a map of treatment possibilities for MS.

FIG. 5 shows a report provided by systems and methods of the invention.

FIG. 6 shows a machine learning system according to certain embodiments.

FIG. 7 shows machine learning calls in newly diagnosed MS individuals.

FIG. 8 shows the magnitude of fold-change differences across mRNA and lncRNA.

FIG. 9A shows a first part of a table of levels of differential expression.

FIG. 9B shows the second part of the table of levels of differential expression.

FIG. 10 shows the machine learning classification of MS using mRNA.

FIG. 11 shows the machine learning classification of MS using annotated lncRNA.

FIG. 12 gives probability calls from machine learning experiments.

FIG. 13 compares accuracy of machine learning methods as binary classifiers.

FIG. 14 illustrates the design of ‘hybrid classifier’.

FIG. 15 shows a proposed model for use of machine learning.

### DETAILED DESCRIPTION

Methods and kits of the invention relate to identifying the presence or risk of disease based on a patient's insurance claims data. Insurance claims data provide a wealth of patient information that can be mined for patterns indicative of disease. By training machine learning algorithms on the insurance claim data of patients with known disease outcomes, those patterns can be identified and then used to classify test patients with unknown outcomes. Trained machine learning algorithms can then quickly identify patients with specific, potentially hard to diagnose diseases by combing the mass amounts of claims data generated every day across the world. The algorithms can catch misdiagnosed patients, saving time and money in their treatment or, depending on the disease outcomes the algorithms are trained on, may be used to identify increased risk of disease prior to onset, grade disease progression, or even predict treatment response. By providing accurate and early diagnoses of degenerative diseases such as MS, methods of the invention allow for earlier and better treatment of the disease, prolonging life expectancies, increasing patients' quality of life, and avoiding unnecessary or harmful treatment.

Any disease, including neurological diseases, inflammatory diseases, rheumatic diseases, and autoimmune diseases may be examined using methods of the invention. In various embodiments, methods of the invention provide for diagnosis of diseases such as multiple sclerosis (MS), Parkinson's disease, Alzheimer's disease, epilepsy, Crohn's disease, ulcerative colitis, IBD (inflammatory bowel disease), systemic lupus erythmatosus, rheumatoid arthritis, and fibromyalgia through analysis of insurance claims data. In certain embodiments, systems and methods may be used to diagnose or monitor forms of cancer, infections, genetic disorders, traumatic brain injury, chronic traumatic encephalopathy, heart disease, diabetes, or endocrine disorders. Systems and methods of the invention may be used to diagnose or monitor injuries such as fractures or injuries to muscle, cartilage, tendons, or ligaments including tears, strains, sprains, or deterioration. Insurance claims data, unlike biopsies or blood draws, is generated by default as a byproduct of medical interactions. Accordingly, general screens of patients' insurance claim data can be implemented without adversely affecting the patients or requiring additional effort or actions on their part.

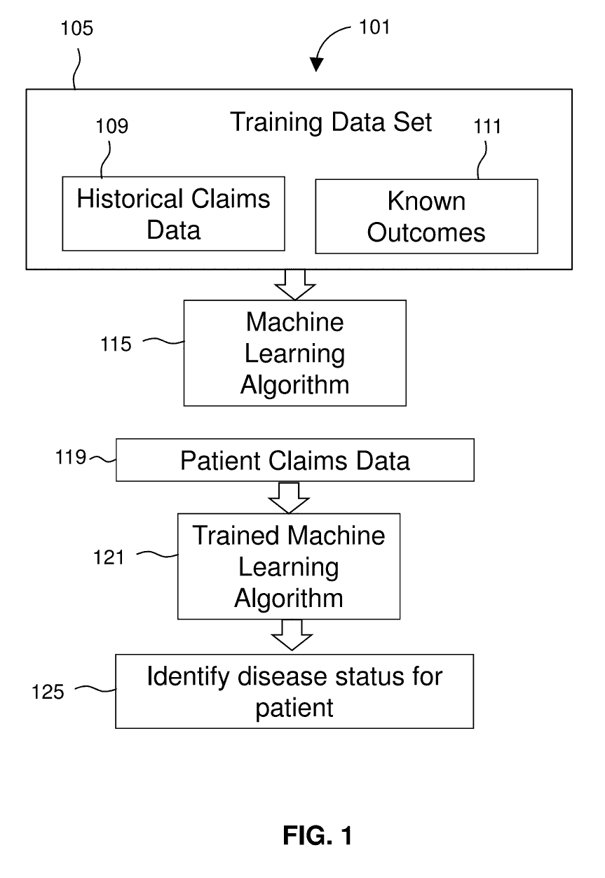

FIG. 1 shows a treatment support method 101according to the invention. A machine learning algorithm 115 is trained on a training data set 105 comprising historical claims data 109 and known outcomes 111. The trained machine learning algorithm 121 is then provided with patient claims data 119, the trained machine learning algorithm 121 then identifying 125 a disease status for the patient.

In various embodiments, the disease status may include identifying a patient at risk of developing a disease. An advantage of the present invention is the ability to identify at-risk patients before the onset of a disease. Once patients having an increased risk of developing a disease are identified, they may be subjected to more rigorous or more frequent screening for the disease so that development of the disease can be caught early and treated quickly. In certain embodiments, a patient identified as being at increased risk of developing a disease may receive preventative treatments targeted at preventing or delaying the eventual development of the disease.

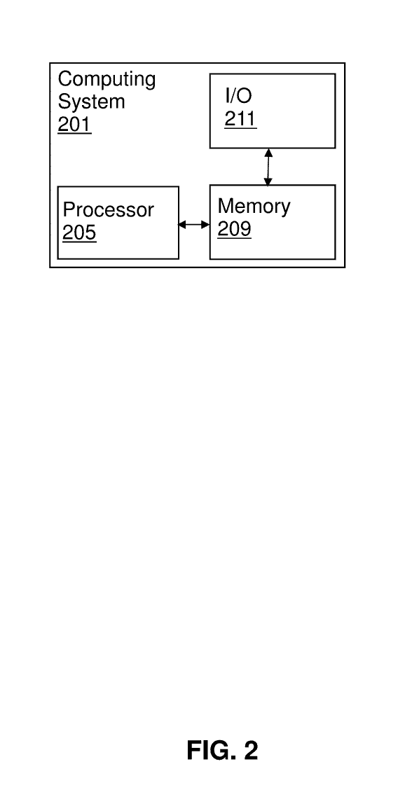

FIG. 2 shows a computing system 201 useful for implementing machine learning algorithms of the invention. The computing system 201 comprises at least one processor 205 coupled to a tangible, non-transitory memory subsystem 209. The computing system 201 may further comprise an input/output device 211.

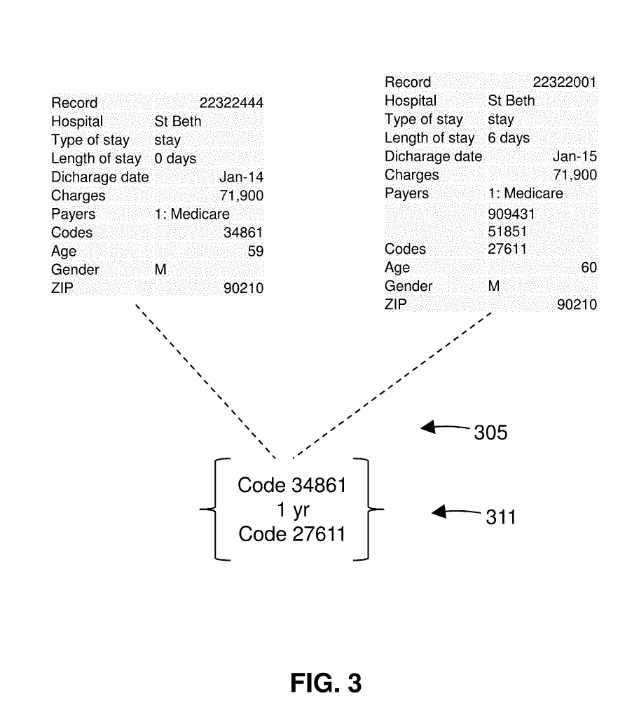

FIG. 3 shows one example of a machine learning system 201 implementing the machine learning algorithm 115 discovering 115 associations in the data. In the depicted embodiment, the system has read 305 from two different medical records and observed the co-occurrence of two different diagnostic codes (34861 and 27611) within a 1 year span for a patient. The system 201 has observed this co-occurrence a number of times that is greater than the number that would be observed if those codes co-occurred within that time span only at random. The system creates an object 311 representing that the co-occurrence has been learned.

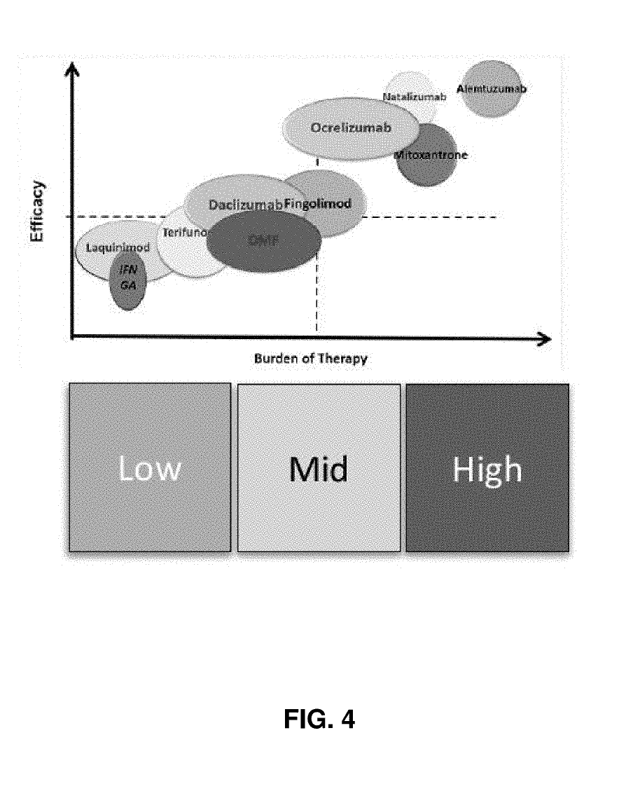

In certain embodiments, identification of a disease may include classifying activity level of a disease in a patient or otherwise grading disease progression. For example, multiple sclerosis (MS) patients can be classified by low, mid, or high disease activity levels as shown in FIG. 4. Further as shown in FIG. 4, treatments have different risk and reward profiles, and treatment decisions should be informed by the patient's specific disease activity level so that higher risk treatments are reserved for patients with high disease activity.

The known patient outcomes provided to the machine learning algorithm may be, for example, a simple diagnosis (e.g., the patient was confirmed positive for a disease), a known disease activity level, or a known response to a specific treatment. Depending on the outcomes provided to the machine learning algorithm, the trained algorithm can then be used to identify patterns indicative of the various outcomes and then to determine a likelihood of a test patient having that outcome based on claims data alone. Where the algorithm is trained on treatment outcomes, it can then be used to predict a test patient's responsiveness to various specific therapies. Accordingly, methods may include recommending a treatment based in part on the prediction where a certain treatment will only be recommended for patients likely to respond thereto.

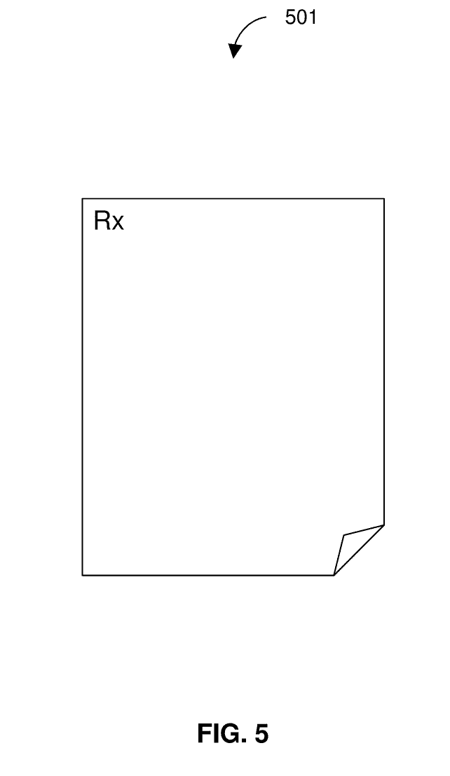

FIG. 5 shows a report 501 with a recommended treatment. A report 501 may take any suitable format. For example, in certain embodiments, the report is an electronic document that is both human-readable and machine-readable, such as a PDF with text-searchable fields or an XML document shared within a system that applies style sheets for display. The report 501 may include information identifying a patient, a disease, and a recommended treatment. For example, the report may predict an individual's responsiveness to a recommended treatment. In certain embodiments, the recommended treatment may be provided in a written report for the patient or a treating physician. In some embodiments, the treatment may be prescribed for the patient or administered to the patient.

As noted above, treatment decisions may also be informed by the patient's specific disease activity level so that higher risk treatments are reserved for patients with high disease activity. For example, where the disease is MS, various treatments have risk/reward or burden/efficacy profiles as shown in FIG. 4. Methods of the invention may include recommending, prescribing, or administering treatments based on the determination of disease activity level by the trained machine learning algorithm. Where the activity level is low, the treatment may include administration of low burden/risk treatments such as laquinimod or teriflunomide. Where the activity level is mid or middle, the treatment may include administration of medium burden/risk treatments such as daclizumab, fingolimod, DMF, or ocrelizumab. Where the activity level is high, the treatment may include administration of higher burden/risk treatments such as ocrelizumab, natalizumab, mitoxantrone, or alemtuzumab.

In certain embodiments, methods of the invention may be used to determine unique patterns or signatures in insurance claim data associated with specific diseases.

Insurance claim data may include Healthcare Common Procedures Coding System (HCPCS), Current Procedural Terminology (CPT), or International Classification of Diseases (ICD) Clinical Modifications (CM), National Drug Codes (NDCs), International Classification of Primary Care (ICPC), or International Classification of Functioning, Disability and Health (ICF) codes for example. Data may include, for example, patient diagnoses, procedures, prescribed therapies, symptoms, geographic location, demographic information, and/or provider information and can be provided with associated chronological data. Claims data can be provided by medical providers or insurers for analysis.

By comparing claims data for healthy and diseased patients, one can identify patterns in the data that are indicative of certain diseases or disease outcomes. In certain embodiments, the claims data and associated known outcomes may be subjected to machine learning analysis to identify patterns most predictive of disease.

In certain embodiments, analytical devices, such as biosensors, may be used to collect, monitor and convey physiological data using the systems and methods described herein. Suitable biosensors include, for example, electrochemical, thermometric, heartrate, optical, piezoelectric, gravimetric, blood glucose, or pyroelectric biosensors that may be used at home or in a clinic. In other embodiments, biosensors may be wearable. Suitable wearable biosensors include, for example, wearable biosensors in a smartwatch, such as the smartwatch sold under the trademark APPLE WATCH, or wearable biosensors in an activity tracker, such as the activity tracker sold under the trademark FITBIT. In embodiments of the invention, analytical devices may be used for conveying diagnostic or prognostic information determined using the systems and methods described herein.

In certain embodiments, methods such as color coded reporting may be used for conveying diagnostic or prognostic information determined using the analytical systems and methods described herein. Analytical devices may be used for conveying the color coded reporting described herein. In order to simplify diagnostic information, specific codes that are indicative of suggested action may be used. For example, a blue color may be used to indicate a low level of risk wherein no action need be taken. A green color may indicate a slightly increased level of risk wherein medical intervention, such as additional testing, should be sought at the patient's convenience. Such an indication may trigger more expensive and/or invasive traditional diagnostic analysis such as a biopsy for example. A red color may be used to indicate a high level of risk or an emergency in which the patient should seek immediate medical attention. The above colors are provided as exemplary indicators and the number and style of the indicator codes may change as one of skill in the art would see fit. For a more nuanced system for example, 5, 10, 15, or more separate indicator codes may be used. Colors, shapes, numbers, letters, or other symbols can be used to convey diagnostic information and recommended action.

Diagnostic and prognostic information such as the aforementioned codes may be provided via a care management system used to monitor or track identified patterns or signals (e.g., insurance claims data, conventional diagnostic imaging, or social data) over time and provide alerts when various thresholds are passed. Analytical devices, such as the biosensors described herein may be used to collect physiological, diagnostic and prognostic information, which may be analyzed with, for example, insurance claims data, social data, and diagnostic data to monitor or track identified patterns or signals over time and provide alerts when various thresholds are passed. The information may be transmitted to the care management system. Alerts may be provided to the patient via the analytical device and to the clinic via the care management system. In certain embodiments, the monitoring may include monitoring adherence to treatment protocols and the alerts may include reminders to comply with treatment. In other embodiments, the monitoring may include treatment efficacy.

Machine learning algorithms may be trained by providing the training data set to the machine learning algorithm and optimizing parameters of the machine learning algorithm until the machine learning algorithm produces output describing the known outcomes.

Any machine learning algorithm may be used to analyze RNA differential expression levels including, for example, a random forest, a support vector machine (SVM), or a boosting algorithm (e.g., adaptive boosting (AdaBoost), gradient boost method (GSM), or extreme gradient boost methods (XGBoost)), or neural networks such as H2O.

Machine learning algorithms generally are of one of the following types: (1) bagging, (2) boosting, or (3) stacking. In bagging, multiple prediction models (generally of the same type) are constructed from subsets of classification data (classes and features) and then combined into a single classifier. Random Forest classifiers are of this type. In boosting, an initial prediction model is iteratively improved by examining prediction errors. Adaboost.M1 and eXtreme Gradient Boosting are of this type. In stacking models, multiple prediction models (generally of different types) are combined to form the final classifier. These methods are called ensemble methods. The fundamental or starting methods in the ensemble methods are often decision trees. Decision trees are non-parametric supervised learning methods that use simple decision rules to infer the classification from the features in the data. They have some advantages in that they are simple to understand and can be visualized as a tree starting at the root (usually a single node) and repeatedly branch to the leaves (multiple nodes) that are associated with the classification.

Random forests use decision tree learning, where a model is built that predicts the value of a target variable based on several input variables. Decision trees can generally be divided into two types. In classification trees, target variables take a finite set of values, or classes, whereas in regression trees, the target variable can take continuous values, such as real numbers. Examples of decision tree learning include classification trees, regression trees, boosted trees, bootstrap aggregated trees, random forests, and rotation forests. In decision trees, decisions are made sequentially at a series of nodes, which correspond to input variables. Random forests include multiple decision trees to improve the accuracy of predictions. See Breiman, L. Random Forests, Machine Learning 45:5-32 (2001), incorporated herein by reference. In random forests, bootstrap aggregating or bagging is used to average predictions by multiple trees that are given different sets of training data. In addition, a random subset of features is selected at each split in the learning process, which reduces spurious correlations that can results from the presence of individual features that are strong predictors for the response variable.

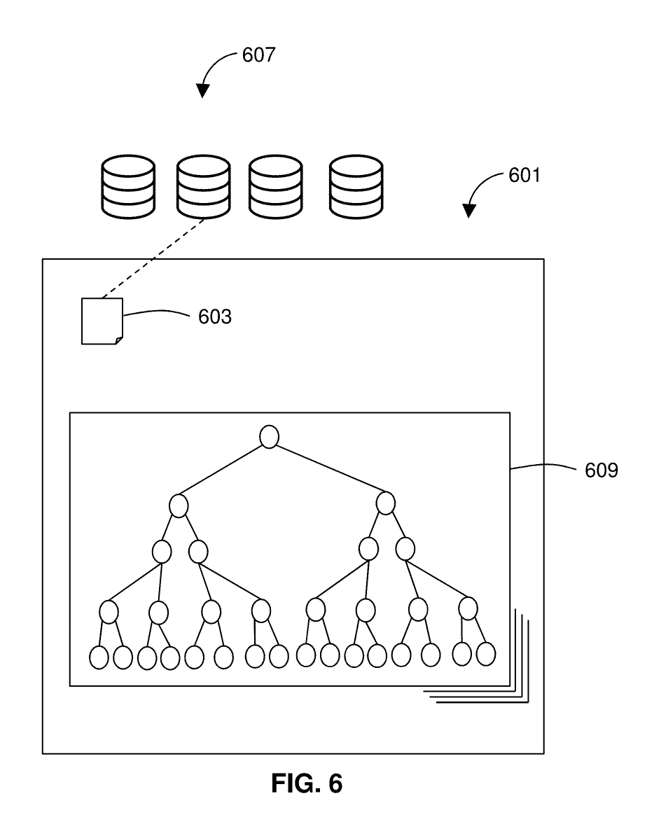

FIG. 6 shows a machine learning system 601 according to certain embodiments using a random forest. The machine learning system 601 accesses data from a plurality of sources 607. Any suitable source of clinical data 607 may be provided to the machine learning system 601. Generally, clinical data includes data that is collected during the course of ongoing patient care or as part of a formal clinical trial program. Types of clinical data include health records/medical records, administrative data, claims data, patient or disease registries, health surveys, clinical trial data, and test results such as clinical laboratory assay results.

In preferred embodiments, the plurality of data sources 607 feed into the machine learning system 601. Any suitable machine learning system 601 may be used. In the depicted embodiment, the machine learning system 601 includes a random forest 609.

The machine learning system 601 may access data from the plurality of sources 607 in any suitable format including, for example, as summary tables (e.g., formatted as comma separated values) or in whole EMR (e.g., to be parsed by a script such as in Perl or SQL in the machine learning system 601). However the initial format, the data ultimately can be understood to include a plurality of entries 603. Each entry preferably includes a datum, or a value, that provides information to the system 601. The value may be a numerical value or it may be a string, such as a classification of disease code (e.g., ICD-9 code or ICD-10 code), which may be aggregated from different sources.

Most preferably, each entry 603 in the data is: specific to one patient from the population, and assigned to a pre-defined category. It will be understood that the data sources 607 may provide anonymized data. In such cases, each entry 603 is preferably specific to a patient and tracked to that patient by a patient ID value, which may be a random string or code. The external data sources 607 may provide the patient ID, or the machine learning system 201 may assign a patient ID to each entry 603. Each entry 603 preferably also has a category. For example, where a data entry 603 is an ICD-9 code, the category may be “ICD-9 Code” (and the value for the entry 603 is the ICD-9 code). In another example, where a data source 607 is an RNA-Seq assay for expression levels, a data entry 603 may be categorized as an expression level for one specific RNA and the value may be the expression level of that RNA. In yet one other example, where a data entry 603 is a patient's weight, the category may be “weight” and the value may be a mass in pounds or kilograms. The machine learning system 601 access the plurality of data sources 607 and discovers associations therein.

SVMs can be used for classification and regression. When used for classification of new data into one of two categories, such as having a disease or not having a disease, a SVM creates a hyperplane in multidimensional space that separates data points into one category or the other. Although the original problem may be expressed in terms that require only finite dimensional space, linear separation of data between categories may not be possible in finite dimensional space. Consequently, multidimensional space is selected to allow construction of hyperplanes that afford clean separation of data points. See Press, W. H. et al., Section 16.5. Support Vector Machines. Numerical Recipes: The Art of Scientific Computing (3rd ed.). New York: Cambridge University (2007), incorporated herein by reference. SVMs can also be used in support vector clustering. See Ben-Hur, A., et al., (2001), Support Vector Clustering, Journal of Machine Learning Research, 2:125-137.

Boosting algorithms are machine learning ensemble meta-algorithms for reducing bias and variance. Boosting is focused on turning weak learners into strong learners where a weak learner is defined to be a classifier which is only slightly correlated with the true classification while a strong learner is a classifier that is well-correlated with the true classification. Boosting algorithms consist of iteratively learning weak classifiers with respect to a distribution and adding them to a final strong classifier. The added classifiers are typically weighted in based on their accuracy. Boosting algorithms include AdaBoost, gradient boosting, and XGBoost. Freund, Yoav; Schapire, Robert E (1997). “A decision-theoretic generalization of on-line learning and an application to boosting”. Journal of Computer and System Sciences. 55: 119; S. A. Solla and T. K. Leen and K. Muller. Advances in Neural Information Processing Systems 12. MIT Press. pp. 512-518; Tianqi Chen and Carlos Guestrin. XGBoost: A Scalable Tree Boosting System. In 22nd SIGKDD Conference on Knowledge Discovery and Data Mining, 2016; the contents of each of which are incorporated herein by reference.

Bayesian networks are probabilistic graphical models that represent a set of random variables and their conditional dependencies via directed acyclic graphs (DAGs). The DAGs have nodes that represent random variables that may be observable quantities, latent variables, unknown parameters or hypotheses. Edges represent conditional dependencies; nodes that are not connected represent variables that are conditionally independent of each other. Each node is associated with a probability function that takes, as input, a particular set of values for the node's parent variables, and gives (as output) the probability (or probability distribution, if applicable) of the variable represented by the node. See Charniak, E. Bayesian Networks without Tears, AI Magazine, p. 50, Winter 1991.

Neural networks, that are modeled on the human brain, allow for processing of information and machine learning. Neural networks include nodes that mimic the function of individual neurons, and the nodes are organized into layers. Neural networks include an input layer, an output layer, and one or more hidden layers that define connections from the input layer to the output layer. Systems and methods of the invention may include any neural network that facilitates machine learning. The system may include a known neural network architecture, such as GoogLeNet (Szegedy, et al. Going deeper with convolutions, in CVPR 2015, 2015); AlexNet (Krizhevsky, et al. Imagenet classification with deep convolutional neural networks, in Pereira, et al. Eds., Advances in Neural Information Processing Systems 25, pages 1097-3105, Curran Associates, Inc., 2012); VGG16 (Simonyan & Zisserman, Very deep convolutional networks for large-scale image recognition, CoRR, abs/3409.1556, 2014); or FaceNet (Wang et al., Face Search at Scale: 80 Million Gallery, 2015), each of the aforementioned references are incorporated by reference.

Regression analysis is a statistical process for estimating the relationships among variables such as features and outcomes. It includes techniques for modeling and analyzing relationships between a multiple variables. Specifically, regression analysis focuses on changes in a dependent variable in response to changes in single independent variables. Regression analysis can be used to estimate the conditional expectation of the dependent variable given the independent variables. The variation of the dependent variable may be characterized around a regression function and described by a probability distribution. Parameters of the regression model may be estimated using, for example, least squares methods, Bayesian methods, percentage regression, least absolute deviations, nonparametric regression, or distance metric learning.

For example, where the disease is MS, methods may include prescription or administration of ocrelizumab, beta interferons, glatiramer acetate, dimethyl fumarate, fingolimod, teriflunomide, natalizumab, alemtuzumab, or mitoxantrone. Where the disease is RA, methods may include prescription or administration of physical therapy, anti-inflammatories, steroids, or immunosuppressive drugs. Where the disease is FMS, methods may include prescription or administration of pain medication, nerve blocking, muscle relaxants, or a selective serotonin reuptake inhibitor (SSRI). Where the disease is SLE, methods may include prescription or administration of steroids or immunosuppressive therapies.

In certain embodiments of the invention, inputs into a machine learning algorithm are scaled or normalized to facilitate meaningful comparisons across categorically different input types. Scaling and Normalization Methods are included. Scaling is used to divide each individual's data by a number to achieve some goal e.g., so that range of values for all data lies in some interval, say, [0,1].

Scaling details may include choices such as “none”, “centering”, “autoscaling”, “rangescaling”, “paretoscaling” (by default=“autoscaling”). A number of different scaling methods are provided: “none”: no scaling method is applied; “centering”: centers the mean to zero; “autoscaling”: centers the mean to zero and scales data by dividing each variable by the variance; “rangescaling”: centers the mean to zero and scales data by dividing each variable by the difference between the minimum and the maximum value; “paretoscaling”: centers the mean to zero and scales data by dividing each variable by the square root of the standard deviation. Unit scaling divides each variable by the standard deviation so that each variance equal to 1.

Normalization details are included and may be used. Normalization is used to divide or shift the total dataset to meet some goal I the overall look of the dataset. For example, one could use the z-score of the data points: (z-μ)/σ. This normalization is determined by the mean of the data and its variance.

A number of different normalization methods are provided: “none”: no normalization method is applied; “pqn”: Probabilistic Quotient Normalization is computed as described in Dieterle, 2006, Probabilistic Quotient Normalization as Robust Method to Account for Dilution of Complex Biological Mixtures. Application in 1H NMR Metabonomics, Anal Chem 78(13):4281-4290, incorporated by reference; “sum”: samples are normalized to the sum of the absolute value of all variables for a given sample; “median”: samples are normalized to the median value of all variables for a given sample; “sqrt”: samples are normalized to the root of the sum of the squared value of all variables for a given sample.

### EXAMPLES

### Example

Some embodiments provide methods for identifying a disease status in a patient from training data that includes claims data and expression levels for RNA such as long non-coding RNA (lncRNA). A machine learning algorithm may be trained to report that a given patient is possibly affected by a disease, and the machine learning algorithm may be able to do so long before disease symptoms manifest to a problematic degree. The machine learning algorithm may be able to give an early warning that a patient is at a high risk of disease based principally on inputs provided in the form of insurance claims data. The insurance claims data may include patterns of diagnoses, treatments, hospital and doctor visits, as well as demographic and geographic data in which latent patterns are predictive of disease risk. The expression level data may be obtained from a blood test. The machine learning algorithm discovers patterns within training data sets in which the training data includes historical claims data, RNA expression levels, and known disease outcomes. The machine learning algorithm may potentially identify a patient at a high risk of disease long before the risk would be discovered by a patient him- or herself, or in the course of routine doctor visits.

Aspects provide a treatment support method that includes training a machine learning algorithm on a training data set that includes historical claims data, expression data, and known outcomes; providing claims data for a patient; and identifying, by the machine learning algorithm, a disease status for the patient. Approximately 10,000-15,000 new diagnoses of multiple sclerosis [MS] are made in the United States each year. Misdiagnosis of MS is costly. A therapeutic strategy that offers the best chance of preserving brain and spinal cord tissue early in the disease course needs to be widely accepted. Early intervention is vital. Methods provide a blood-based test able to both confirm and monitor MS patients. Methods use the potential for lncRNA expression levels analyzed with machine learning to not only classify MS but also indicate treatment responses. RNA-based testing platform starting at the point of blood collection, may include shipping a blood specimen to a clinical lab, sample processing, and reporting of test results to a healthcare provider. Methods may use a machine learning approach and gene expression-based algorithm measuring lncRNA species in whole blood for a discriminatory test for identifying inflammatory diseases including multiple sclerosis as well as monitoring patient responses to therapy.

Autoimmune diseases manifest over a long period of time during which patients are asymptomatic. Elucidation of lncRNAs as actionable genomic biomarkers allows early indications of unregulated, potentially destructive autoimmune processes. Methods use measurements of novel lncRNAs in whole blood in a test that is bifunctional allowing both diagnostic confirmation and monitoring of patients diagnosed with multiple sclerosis.

lncRNAs are recently discovered regulatory RNA molecules that do not code for proteins but influence a vast array of biological processes. lncRNAs exhibit greater cell-type specific patterns of expression than protein-coding genes. For example, cells as similar as the double negative stages of thymocyte development, DN1, DN2, DN3, and DN4, express many more unique lncRNAs than unique protein-coding genes. In methods herein, disease-associated lncRNAs exhibit far greater differences in expression than disease-associated mRNAs. Here, lncRNAs are biomarkers of human disease. Using measured expression of mRNAs and annotated lncRNAs in MS, healthy controls, and disease control subjects, machine learning classifiers are constructed for distinguishing multiple sclerosis from other diseases and healthy controls. Both mRNA and annotated lncRNA datasets were used as inputs into these classifiers and standard calculations of accuracy, sensitivity, and specificity are used to determine the effectiveness of both approaches to correctly classify MS using RNA data.

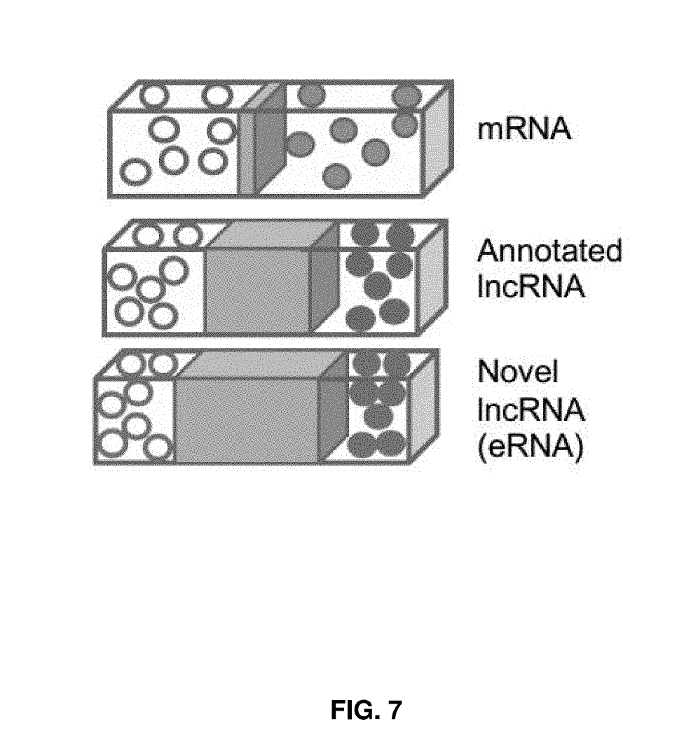

FIG. 7 shows the separation of machine learning calls in newly diagnosed MS individuals versus non-MS (healthy controls or disease controls) using methods of the disclosure. As shown, machine learning gives separation of probability calls for newly diagnosed MS patients using mRNA versus annotated lncRNA or novel lncRNA data. Machine-learning algorithms include binary classifiers that can be viewed as a box with a dividing plane down the middle. Each ball represents a control (open circles) or case (closed circles). The mRNA- and lncRNA-based tests of the disclosure have about 90% accuracy. The gray box with accompanying open (control) and closed (newly diagnosed MS cases) circles illustrates that a lncRNA-based diagnostic test has a greater distance between all controls (open circles) and all cases (closed circles). Methods use novel lncRNA datasets for maximum separation between cases and controls. To extend analysis of RNAs differentially expressed in MS, methods use RNA-sequencing to identify novel lncRNAs. There are about 20,000 genes that encode annotated lncRNAs in the human genome. The annotated lncRNAs are identified, curated and predicted to be non-coding by computational analysis. Novel lncRNAs are determined using de novo RNA sequencing pipelines. The novel lncRNAs are typically >200 base pairs in length, do not code for protein, lack conventional promoters, are transcribed from transcriptional enhancers, and are poly-adenylated. Early results suggest that these lncRNAs exhibit profound differences in MS versus CTRL and support the notion that lncRNA expression data has discriminatory power for disease prediction and diagnosis.

The annotated lncRNA datasets exhibit differences of 4-fold or greater whereas the mRNA datasets have few targets with greater than a two-fold change in the patient population we examined. Machine learning is able to capture these larger expression differences. The probability score is essentially a confidence score that the computer uses to distinguish case/control comparisons. Higher probability scores indicate that the computer is more confident that a patient groups with others of a certain condition. It may be that greater differences in expression among MS patients observed using lncRNA datasets increases resolution of the machine learning probability calls to permit tracking of treatment responses. The disclosure includes a machine learning model for these novel lncRNA data. Methods include whole genome RNA-sequencing data to identify mRNAs, known or annotated lncRNAs, and novel lncRNAs (eRNAs) differentially expressed in whole blood obtained from CTRL subjects and subjects with MS: MS-CIS (subjects with clinical symptoms consistent with MS who received a formal diagnosis of MS at a later date, usually within one year), MS-NAIVE (subjects at their initial diagnosis of MS but before onset of therapies), and MS-EST (subjects with established MS of 1-3 years duration, note that MS-EST subjects were not on beta interferon).

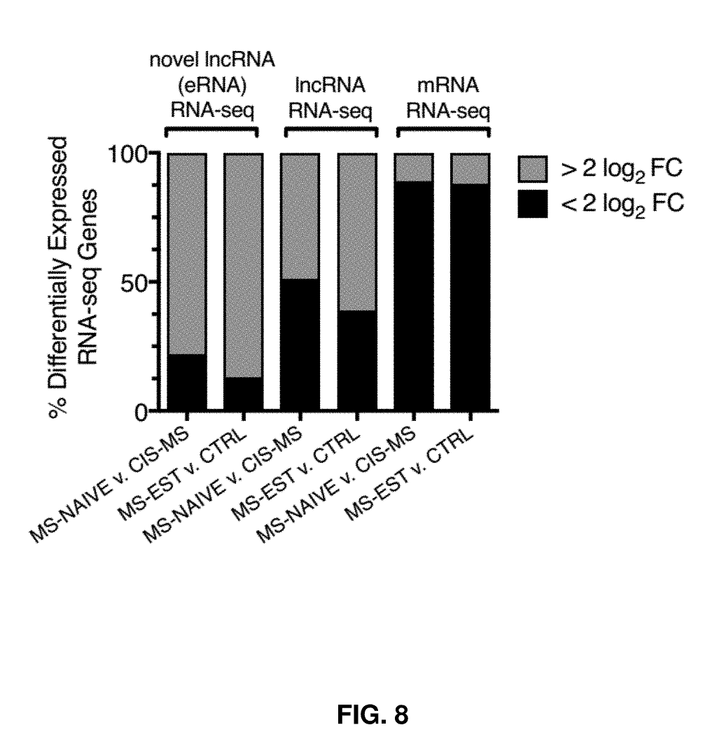

FIG. 8 shows the magnitude of fold-change differences across mRNA and lncRNA genes at distinct stages of multiple sclerosis. Plots are the percentage of differentially expressed (DE) genes as a function of >2 or <2-fold change expression ratios, log2, across eRNAs (novel lncRNAs; left), annotated lncRNAs (middle) and mRNAs (right). Differentially expressed genes all have an adjusted p value <0.05 across two experimental comparisons: (1) MS-NAIVE versus CIS-MS and (2) MS-established (MSEST) versus healthy control (CTRL) subjects. Comparison of the log2 fold-change differences in healthy control versus MS-EST found 3,253 novel RNAs, 1,859 differentially expressed mRNAs and 752 annotated lncRNAs. In the MS-NAIVE versus the MS-CIS cohort, 1,729 novel RNAs, 149 annotated lncRNAs, and 818 mRNAs were differentially expressed. Differences in expression of novel lncRNAs ranges in magnitude from 23 to 26 or 8-fold to 64-fold, annotated lncRNAs ranges in magnitude from 22 to 24 or 4-fold to 32-fold in the different cohorts while differences in expression of mRNAs are typically <22 or <4-fold. Additional analysis of the differentially expressed novel lncRNAs, annotated lncRNAs and mRNAs assessed using DESeq2 found that, on average, >50% of novel lncRNAs and annotated lncRNAs in the MS-NAIVE versus MS-CIS and MS-EST versus CTRL cohorts, respectively, have greater than a 4-fold change in gene expression. Thus, differential expression of the novel lncRNAs in MS is greater than expression differences observed in either annotated lncRNAs or mRNAs.

Candidate annotated lncRNAs that are differentially expressed between one, two or three MS cohorts and CTRL are identified. Targets are determined by selecting the maximum difference in expression, log2, smallest q-value, and required average expression levels in MS and CTRL to be greater than 0.05 FPKM. Primer pairs are designed for each candidate lncRNA. The list of candidate annotated lncRNAs may be refined using the following selection criteria: (1) average cycle threshold, Ct, <32 after RNA isolation from a blood sample, cDNA synthesis and PCR amplification, (2) amplicon is a single band detected on agarose gels of the correct size, (3) coefficient of variance <2.0 among multiple replicates (standard deviation/mean) and (4) amplicon sequence verification. Methods identify lncRNAs for which differential expression is measured among MS cohorts and CTRL. Samples are treated as follows: (i) after informed consent, blood is collected from subjects into blood collection tubes, (ii) total RNA is purified using RNA isolation kits sold under the trademark PAXGENE, (iii) RNA amounts are measured using a Nanodrop spectrophotometer, (iv) cDNA synthesis is performed using oligo-dT primers and Superscript 3 (Invitrogen), (v) PCR reactions are performed in 384-well plates in 10 microliter volumes containing 1 ng/μl cDNA, Taqman master mix and SYBR green. Levels of expression of those annotated lncRNAs are compared in the different RRMS cohorts, MS-CIS, MSNAIVE, and MS-EST to CTRL using GAPDH expression for normalization. Results are expressed as the ratio between the disease cohorts and CTRL cohorts, log2. In general, most annotated lncRNAs are under-expressed rather than over-expressed in the MS cohorts compared to CTRL cohorts.

Using RNA-seq, differentially expressed mRNAs are identified in blood in cohorts of CTRL (N=8), MS-CIS (N=6), MS-NAIVE (N=6), MS-EST (N=8). 46 target mRNAs are picked and included GAPDH as a housekeeping gene, designed TLDA (384-well) cards and analyzed expression of those mRNAs in a larger cohort of about 1400 subjects. Those cohorts include healthy controls, disease controls and subjects with MS to identify annotated lncRNA and mRNA expression differences measured by PCR. mRNA targets are determined by selecting the maximum difference in expression, log2, smallest q-value, and required average expression levels in MS and CTRL be greater than 0.05 FPKM. It may be informative to actually compare levels of differential expression of the mRNAs and lncRNAs selected from the RNA-seq experiment in larger cohorts. To do so, a heatmap is constructed to illustrate the level of differential expression of the selected mRNAs and annotated lncRNAs measured by RT-PCR in each MS cohort compared to the CTRL cohort.

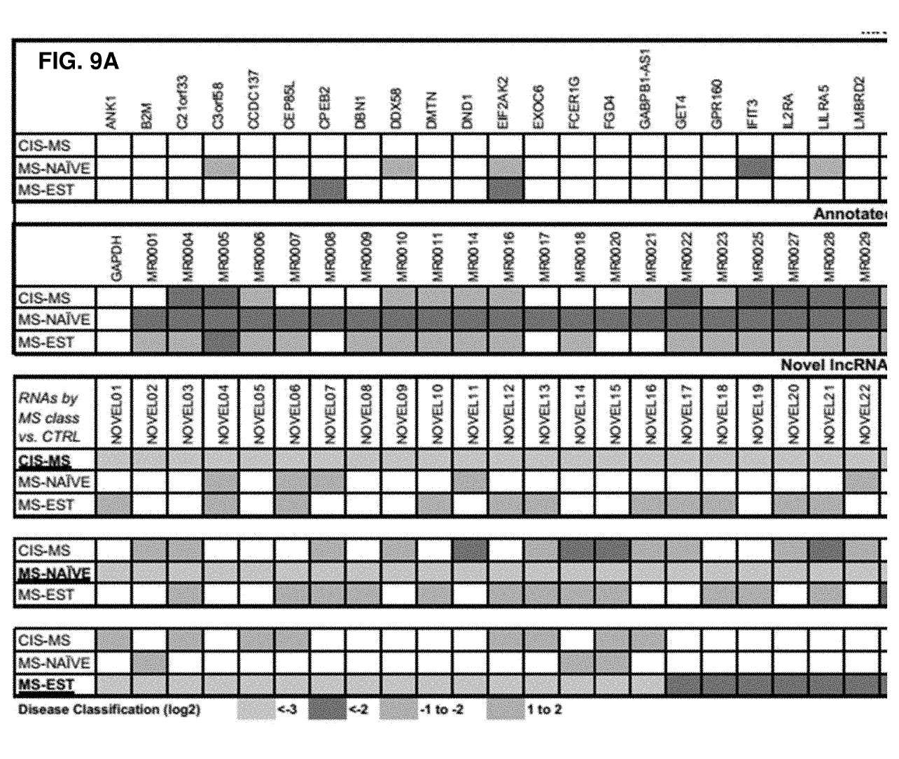

FIG. 9A and FIG. 9B give levels of differential expression of select mRNAs and lncRNAs between indicated MS cohorts and CTRL cohorts. MS cohorts are divided into MS-C, MS-N and MS-E. Results are expressed as mean log2 ratios between cases and controls. Results show that levels of differential expression of these selected annotated lncRNAs in these MS cohorts is greater than the levels of differential expression of the selected mRNAs in those same MS samples.

Gene expression data derived from peripheral whole blood, is used to train and test models capable of distinguishing MS patients from healthy control subjects with no family history of autoimmune disease (CTRL), healthy unaffected family members of patients with MS (CTRL-UFM) and patients with other inflammatory (OND-I) and non-inflammatory (OND-NI) neurologic diseases. The overall accuracy using both datasets were similar with AUC values of ˜0.94 for both mRNA and annotated lncRNA data and overall accuracy levels of 92% using mRNA data and 94% using annotated lncRNA data.

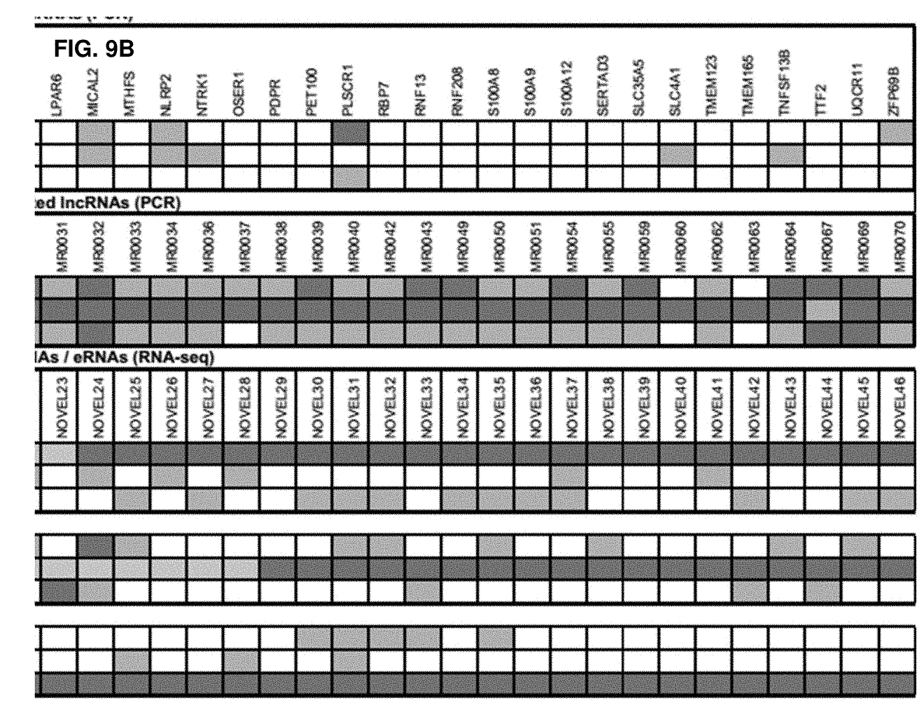

FIG. 10 shows the machine learning classification of MS using mRNA.

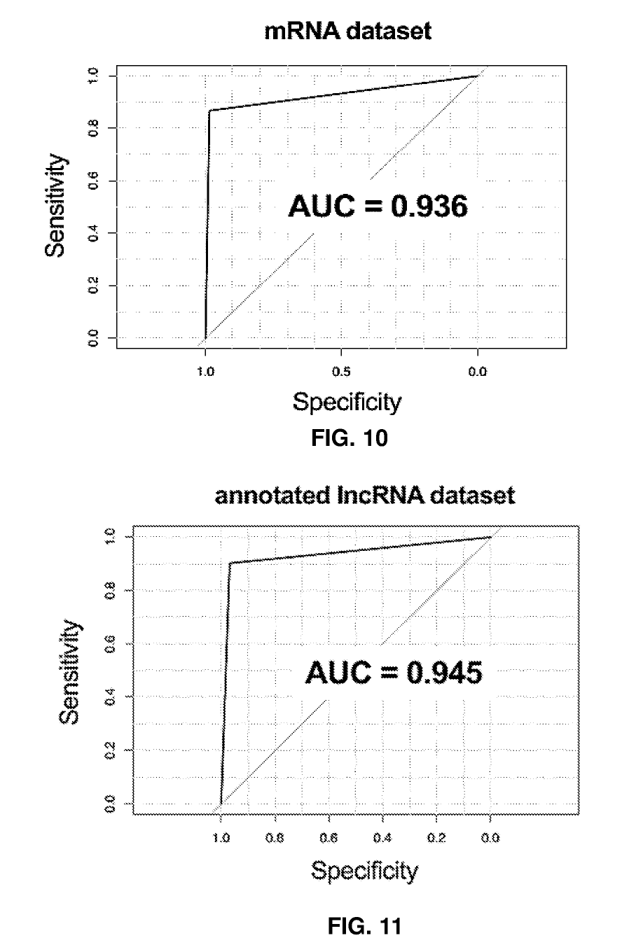

FIG. 11 shows the machine learning classification of MS using annotated lncRNA datasets and probability score distributions for MS patients receiving treatment. Binary classification inputs derived from CTRL, CTRL-UFM, MS, OND-I, and OND-NI subjects are used as inputs to train and test different combinations of machine learning methods capable of multi-class discrimination. FIG. 10 and FIG. 11 give ROC curves and calculated area under the ROC curve values for optimal multi-category classifier combinations capable of discriminating MS for optimal multi-category classifier combinations capable of discrimination vs. non-MS using mRNA or annotated lncRNA data.

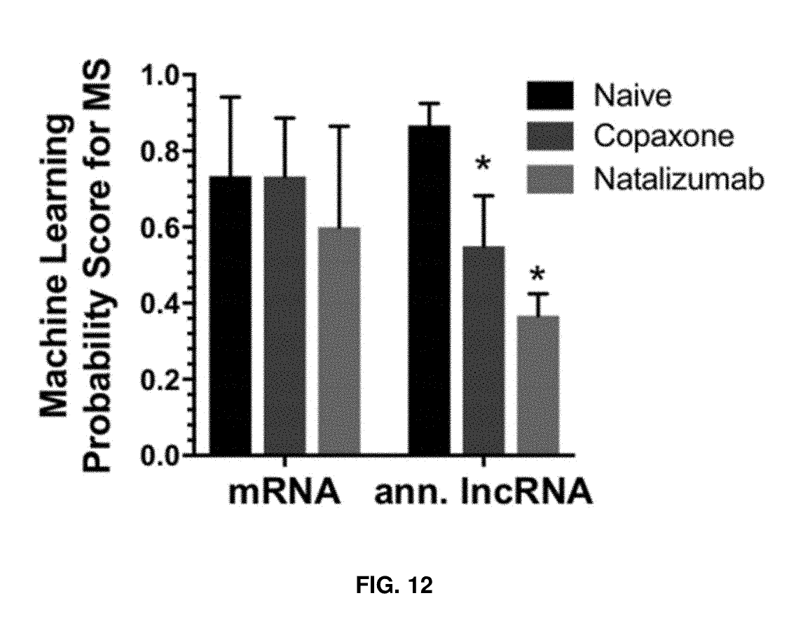

FIG. 12 gives probability calls from machine learning experiments using mRNA or annotated lncRNA datasets. Cross-sectional expression data from patients at the time of diagnosis but before treatment (MS-NAÏVE) and established MS patients (MS-EST) sub-divided into those receiving glatiramer acetate and those receiving natalizumab. Machine learning scores are determined for MS and reported on a scale from 0 to 1. Q-value are determined; * identifies differences statistically significant after correction for false discovery rates using Benjamini-Hochberg correction methods for the indicated group vs. MS-NAIVE.

In MS, one in three patients will change treatments in the first two years of treatment due to increasing disability or relapse. Thus, tools to effectively monitor response to treatment would be clinically useful to accelerate alteration of treatment plans, as needed. Here, mRNAs and lncRNAs deliver similar accuracies when these expression datasets are analyzed using machine learning approaches to classify MS. Use of lncRNA data, however, appears to offer increased resolution in the resulting probability calls among established MS patients receiving treatment compared to patients prior to the initiation of therapy (MS-NAÏVE). Scores reported here were obtained in cross-sectional studies using stable patients receiving treatment for up to 1 year. The greater differences in annotated lncRNA expression among the MS patients allow one to discover changes in the resulting probability scores. The greatest resolution may be found in machine learning probability scores when novel lncRNAs are used. Longitudinal assessment of gene expression will also allow one to correlate these probability scores with clinical measurements of disease activity.

Thus, expression levels of annotated lncRNAs in blood show greater differential expression between cases and controls than mRNAs. The disclosure provides a machine learning classifier capable of accurately distinguishing MS using novel lncRNA data. Machine learning methods may develop discriminatory case/control classifiers using expression of annotated lncRNAs that show dynamic changes in machine learning probability scores when patients initiate treatment. Differences are observed when MS patients are treated with low burden, lower efficacy therapeutics compared to therapeutics that have higher efficacy but are often associated with a higher burden of treatment (worse safety, more difficult administration route). Different machine learning methods such as, ratioscore, support vector machines, adaboost (adaptive boosting), gradient boost method GBM), extreme gradient boost methods (XGBoost), neural networks, and random forest may be used to determine whether novel lncRNA-derived datasets can effectively track clinical responses to treatment.

Collection of patient blood samples is performed in MS patients initiating therapy in distinct treatment groups. Patients are followed and corresponding probability scores determined using the novel lncRNA classification model to correlate resulting RNA-derived scores with clinical assessments that are frequently used in clinical trials to determine drug efficacy

Methods include determining expression levels of target novel lncRNAs (eRNAs) in blood obtained from cohorts of subjects that include 1) subjects with RRMS (MS-CIS, MS-NAIVE, MS-EST), 2) healthy controls, 3) neurologic disease controls including both inflammatory and non-inflammatory disorders, and 4) peripheral autoimmune disease controls.

Determining expression levels of novel lncRNAs in blood in a cohort of ˜1600 subjects will satisfy the need for sufficient power, geographic distribution, and inclusion of other disease controls. The expression data are used to construct a machine learning classifier capable of identifying MS using gene expression inputs.

Primary progressive multiple sclerosis (PPMS) is a form of multiple sclerosis that is characterized by progressive deterioration without periods of relapses and remissions and it is not known if it is an inflammatory or autoimmune disease. Secondary progressive multiple sclerosis (SPMS) is a progression of RRMS when subjects move to a stage of disease that is continuously progressive without periods of remission. Since SPMS is a late stage of RRMS, these subjects will not be included in our analysis as this would represent a totally separate project. The experimental approach is outlined. Blood from volunteers will be collected in tubes to immediately stabilize RNA (PAXGENE tubes have the advantage over other tubes since these have received FDA approval as a method to collect blood for RNA- and DNA-based diagnostic studies). Blood samples are stored at −80 degrees C. until processing. Total RNA is purified using RNA purification kits specifically designed for PAXGENE tubes. Total RNA is reverse transcribed to cDNA using Superscript III First-Strand Synthesis Kit from Invitrogen. Custom designed primer pairs and SyberGreen are used with PCR master-mix. PCR amplification is performed using our ABI QuantStudio 12K Flex instrument. Ct values are downloaded to computer for computational analysis and quantitative expression levels of novel lncRNA transcripts are determined by normalization to GAPDH transcript levels. Of all the proposed ‘housekeeping genes’, e.g. GAPDH, ACTB, B2M, and 18S and 28S rRNA, GAPDH levels exhibit the least variability across all samples.

The novel lncRNA expression data is used as inputs into machine learning classifiers to build classifiers capable of distinguishing MS and monitoring response to treatment.

To construct machine learning classifiers capable of distinguishing MS from other experimental groups using novel lncRNA data and test the hypothesis that longitudinal changes in RNA expression profiles analyzed using machine learning result in MS probability scores that correlate with clinical responses to treatment. Methods will use novel lncRNA datasets to construct a machine learning model capable of classifying MS versus healthy and disease controls. Accuracy, sensitivity, and specificity of this novel lncRNA model for MS will be compared to those we have constructed previously for mRNA or annotated lncRNA datasets outlined in the preliminary studies. Methods may use 46 target genes and 2 GAPDH assays to fit well into 384-plate formats. Ct data (log2) are linearized by either normalizing to GAPDH using the formula 2(Test Gene CT-GAPDH CT) or using the formula 2(41-Test Gene CT). Expression ratios of two genes rather than a single gene may be as inputs (using gene ratios serves to normalize the data without having to assume that a given ‘housekeeping’ gene is consistently expressed at the same level across all samples; also, a ratio of an over-expressed gene and an under-expressed gene produces a greater quantitative difference than a single gene). All possible ratios are calculated, in this format: 48×48=2304, and permutation testing identifies the ‘best’ ratios by randomly selecting 80% of the control group to compare to 80% of the test group and repeating this process 200 times. The smallest number of ratios producing the maximum separation between case and control groups is identified, thus defining the ratio score. Those ratio values are also the input for support vector machines and other machine learning algorithms.

In addition to support vector machines, other machine learning methods including random forest, adaptive boosting (adaboost), gradient boost method (GBM), extreme gradient boost method (XGBoost) and neural networks may be used. Machine learning algorithms generally are of one of the following types: (1) bagging, (2) boosting, and (3) stacking. In bagging, multiple prediction models (generally of the same type) are constructed from subsets of classification data (classes and features) and then combined into a single classifier. Random Forests classifiers are of this type. In boosting, an initial prediction model is iteratively improved by examining prediction errors. Adaboost.M1 and eXtreme Gradient Boosting are of this type. In stacking models, multiple prediction models (generally of different types) are combined to form the final classifier. These methods are called ensemble methods. The fundamental or starting methods in the ensemble methods are often decision trees.

Decision trees are non-parametric supervised learning methods that use simple decision rules to infer the classification from the features in the data. They have some advantages in that they are simple to understand and can be visualized as a tree starting at the root (usually a single node) and repeatedly branches to the leaves (multiple nodes) that are associated with the classification.

Bagging and boosting methods attempt to overcome over-fitting shortcomings. A support vector machine is a classification algorithm derived by a supervised learning algorithm that attempts to partition feature data in high dimensional space by using hyperplanes. Determination of hyperplanes is often performed in a nonlinear fashion using the kernel trick. Some machine-learning methods work best as binary classifiers.

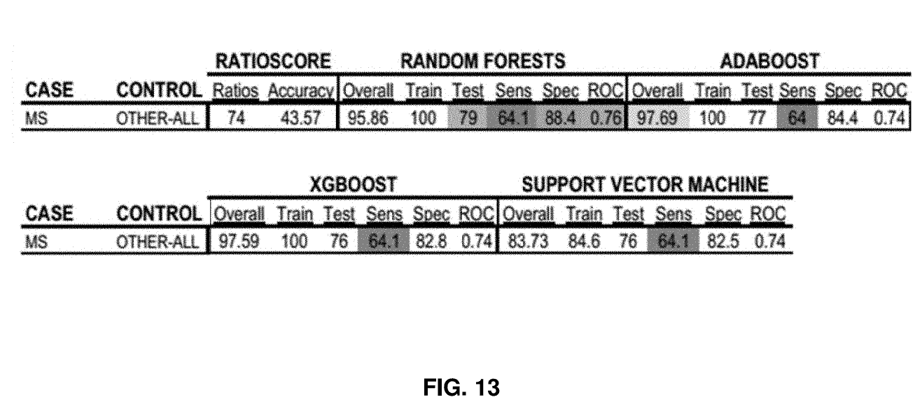

FIG. 13 compares accuracy of machine learning methods as binary classifiers. Cases=all MS subjects and Controls=all nonMS subjects, CTRL, OND-I and OND-NI. Training is performed with 75% of the dataset and validation with an independent dataset representing 25% of the total dataset. Sensitivity, specificity and ROC curves were determined using standard calculations. Therefore, we expanded our approach and considered whether multi-category classifiers could be used to distinguish among the CTRL, MS, and OND classes. We developed a new computational pipeline in what we term a ‘hybrid classifier’ to accomplish this task utilizing principle components of each ratio score output derived from each of the 21 pairwise comparison

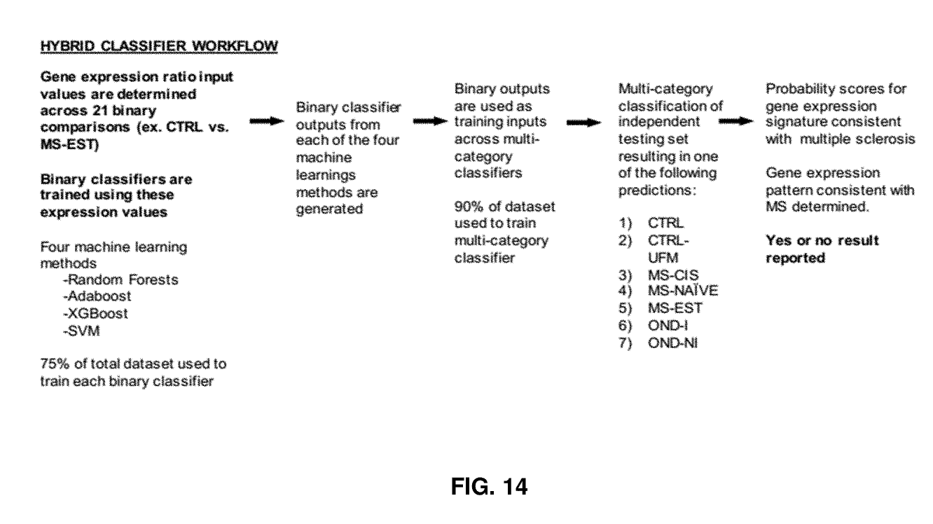

FIG. 14 illustrates the design of ‘hybrid classifier’. The basic idea is to have constructed a series of independent binary classifiers to generate outputs that are evaluated in a second set of binary inputs to create the multi-category classification. Each of the four machine learning methods is constructed with optimal ratio score inputs capable of discriminating between those case/control comparisons for the designated comparator groups. Those algorithms are then trained using ratio score values with 75% of the dataset and tested with 25% of the dataset. These same 21 algorithms are then applied to 90% of the dataset to generate binary inputs across each patient sample. For instance, across the series of the first three comparisons: (1) CTRL vs. CTRL-UFM [CTRL-UFM; healthy controls that are unaffected family members of patients with MS], (2) CTRL vs. CIS-MS, or (3) CTRL vs. MS-NAÏVE, a healthy subject would ideally score as CTRL in each of the three comparisons. A subject with an inflammatory neurologic disorder like optic neuritis, however, might score positively for CTRL in some comparisons but score positively for MS in others as inflammatory neurologic disorders may more closely resemble MS than CTRL. Thus, the series of outputs for each patient according to the binary classifier for 90% of the dataset is determined and then each machine learning method is used to classify a subject according to one of seven classifications: CTRL, CTRL-UFM (control unaffected parents of subjects with multiple sclerosis, 0 CIS-MS, MS-NAÏVE, MS-EST, OND-I, or OND-NI. Each series of machine learning inputs was placed through alternative multi-category classifiers to augment the analysis. For example, SVM inputs were placed through random forests, adaboost, XGBoost, and SVM multicategory classifiers using inputs derived from SVM. In this multi-category classifier, a subject is correctly classified for MS if the gene expression signature is classified into any of three MS classes: MS-CIS, MS-NAÏVE, or MS-EST.

Different combinations of binary inputs with each of the multi-category classifiers didn't dramatically affect overall accuracy. Random forests, adaboost, and XGBoost or a combination thereof led to the best overall validation results with overall accuracy ranging from 88%-94%. ROC curves from the top overall accuracies are reported. Results indicate that a hybrid classifier approach correctly classifies MS subjects from other healthy and disease controls with greater than 90% accuracy using a single algorithm.

Summary of novel lncRNA classifier creation and longitudinal analysis of treatment response: Analysis of novel lncRNA expression data uses machine learning classifiers of various machine learning methods: random forests, adaboost, XGBoost and SVM to evaluate the binary inputs. The resulting multi-category classifier generates probability scores for MS using novel lncRNA expression data from MS patients initiating treatment.

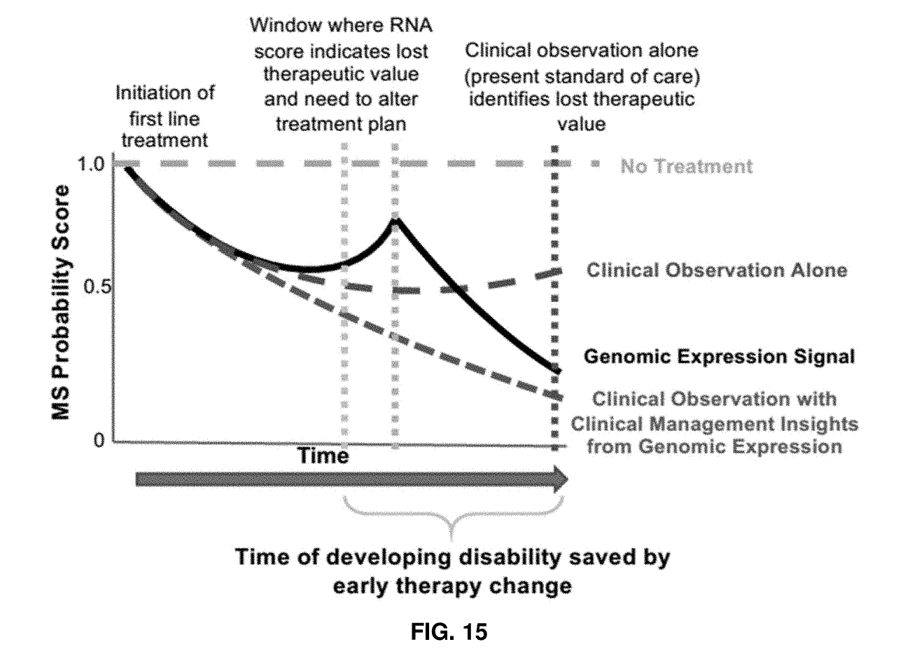

FIG. 15 shows a proposed model for use of machine learning probability scores derived from lncRNA expression data to prevent patient disability and scientific premise, rigor and reproducibility: The proposal is based on work showing that mRNA-based gene expression machine learning classifiers can be developed with the potential of improving and accelerating diagnosis of complex human diseases, including autoimmune diseases. Methods use not only mRNA-based gene expression profiles to build better diagnostics, but to extend analysis of lncRNA expression profiles to better classify autoimmune diseases including multiple sclerosis. mRNA- and lncRNA-based gene expression profiles can be used to determine clinical responsiveness to treatments for MS, based on the fact that lncRNAs seem to exhibit greater cell-type specific expression patterns than canonical mRNAs.

Greater loss or gain of those RNAs may be associated with certain diseases, including MS, that are thought to arise through cell type specific changes in phenotype and these may be controlled by changes in lncRNA expression patterns. Furthermore, those changes may be modulated by therapies that are effective in disease management. It may be that mRNAs and lncRNAs are induced in response to standard treatments of autoimmune disease through cross-sectional analyses.

Machine learning methods are performed using both a training set to train the different algorithms and a totally independent testing set to determine accuracy. Machine learning probabilities for each sample in the independent validation set are generated by the computer along with standard calculations of sensitivity, specificity and ROC curve analysis to determine overall accuracy.

### Incorporation by Reference

References and citations to other documents, such as patents, patent applications, patent publications, journals, books, papers, web contents, have been made throughout this disclosure. All such documents are hereby incorporated herein by reference in their entirety for all purposes.

### Equivalents

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A treatment support method, the method comprising:
training a machine learning algorithm on a training data set that includes historical claims data and known outcomes;
providing claims data for a patient; and
identifying, by the machine learning algorithm, a disease status for the patient.

2. The method of claim 1, wherein identifying the disease status includes identifying the patient as being at a high risk for a disease.

3. The method of claim 1, wherein the machine learning algorithm is implemented in a computing system comprising at least one processor coupled to a tangible, non-transitory memory subsystem.

4. The method of claim 1, wherein identifying the disease status includes classifying an activity level of a disease in the patient.

5. The method of claim 4, further comprising recommending a treatment for the patient.

6. The method of claim 5, further comprising administering the treatment to the patient.

7. The method of claim 6, wherein the disease is multiple sclerosis (MS), and further wherein the activity level is selected from the group consisting of low, middle, and high, and further wherein:
when the activity level is low, the treatment includes the administration of laquinimod or terifunomide;
when the activity level is middle, the treatment includes the administration of daclizumab, fingolimod, DMF, or ocrelizumab; and
when the activity level is high, the treatment includes the administration of ocrelizumab, natalizumab, mitoxantrone, or alemtuzumab.

8. The method of claim 1, wherein identifying the disease status includes determining a therapeutic efficacy of a treatment.

9. The method of claim 1, wherein identifying the disease status includes determining a disease progression.

10. The method of claim 1, wherein the disease is selected from the group consisting of a neurological disease, an inflammatory disease, a rheumatic disease, and an autoimmune disease.

11. The method of claim 1, further comprising training the machine learning algorithm by providing the training data set to the machine learning algorithm and optimizing parameters of the machine learning algorithm until the machine learning algorithm produces output describing the known outcomes.

12. The method of claim 1, wherein the machine learning algorithm includes one selected from the group consisting of: a neural network, a random forest, Bayesian classifier, logistic regression, decision tree, gradient-boosted tree, multilayer perceptron, one-vs-rest, and Naive Bayes, a support vector machine (SVM), and a boosting algorithm.

13. The method of claim 12, wherein the classification model includes a random forest comprising a plurality of decision trees.

14. The method of claim 13, wherein one or more of the decision trees receive parameters selected from the group consisting of: icd9 codes; cpt codes; HCPCS codes; patient demographic data; and patient geographic data.

15. The method of claim 1, wherein the classification model includes a neural network.

16. The method of claim 1, wherein the disease is selected from the group consisting of Parkinson's disease, Alzheimer's disease, and epilepsy.

17. The method of claim 1, wherein the disease is selected from the group consisting of Crohn's disease, ulcerative colitis, and inflammatory bowel disease (IBD).

18. The method of claim 1, wherein the disease is selected from the group consisting of systemic lupus erythmato sus, rheumatoid arthritis, and fibromyalgia.
